(19) United States
(12) Patent Application Publication
(10) Pub. No.: US 2014/0273270 A1
(43) Pub. Date: Sep. 18, 2014

(54) DIRECT TEMPERATURE MEASUREMENT OF A TEST STRIP

(71) Applicant: Polymer Technology Systems, Inc., Indianapolis, IN (US)

(72) Inventors: Xin (Cindy) Wang, Clifton Park, NY (US); Hoi-Cheong Steve Sun, Mount Kisco, NY (US); Paul M. Ripley, Nanuet, NY (US); Mu Wu, Hopewell Junction, NY (US)

(73) Assignee: Polymer Technology Systems, Inc., Indianapolis, IN (US)

(21) Appl. No.: 13/804,824

(22) Filed: Mar. 14, 2013

(51) Int. Cl. G01N 33/72; G01N 33/558

(52) U.S. Cl. CPC G01N 33/726; G01N 33/558
USPC 436/501; 422/69

(57) ABSTRACT

An optical lateral flow fluid analyte testing device includes a test strip having at least one zone for measuring the concentration of a target analyte in a fluid sample. Devices and methods for facilitating a reliable reading are disclosed.

### BACKGROUND OF THE INVENTION

The present invention relates to a fluid analyte device that measures the concentration of one or more target analytes within a sample fluid, and more particularly to a fluid analyte device that facilitates the determination of whether conditions are met to facilitate the taking of an accurate reading.

Fluid analyte systems measure substances found in blood or another body fluid. The quantitative determination of analytes in body fluids is of great importance in the diagnosis and maintenance of certain physiological conditions. In particular, certain diabetic individuals need to frequently check the glucose level in their blood to regulate the glucose intake in their diets. The results of such tests can be used to determine what, if any, medication, e.g., insulin, should be administered to the individual.

High levels of blood glucose cause over-glycation of proteins, including hemoglobin, throughout the body. Glycation of hemoglobin can occur at the amino termini of the alpha and beta chains, as well as other sites with free amino groups. Hemoglobin A undergoes a slow glycation with glucose that is dependent on the time-average concentration of glucose over the 120-day life span of red blood cells. The most prevalent and well-characterized species of glycated hemoglobin A is A1C, making up approximately 3% to 6% of the total hemoglobin in healthy individuals. The correlation of A1C and blood glucose levels make it a useful method of monitoring long-term blood glucose levels in people with diabetes. The mean (average) blood glucose level (MBG) is a function of the A1C levels, and is therefore derivable.

Measurement of blood glucose concentration is typically based on a chemical reaction between blood glucose and a reagent. The chemical reaction and the resulting blood glucose reading as determined by a blood glucose meter is temperature sensitive. Therefore, a temperature sensor is typically placed inside the blood glucose meter to determine the temperature of the blood glucose meter. The calculation for blood glucose concentration in such meters typically assumes that the temperature of the reagent is the same as the temperature reading from a test sensor placed inside the meter. In this regard, instead of using the test strip or reagent temperature in a given algorithm to measure blood glucose concentration, the temperature of the meter or cartridge housing is often utilized. However, if the actual temperature of the reagent and the test meter or cartridge housing are different, the calculated blood glucose concentration may be erroneous.

Moreover, the test sensor may have been stored in a relatively cold or hot environment that is not within the ideal operative range. To maximize the shelf life of some components of the measuring kit, e.g., test cartridges, it may be desirable to store the components in a refrigerated environment, e.g., between 2-8° C. If refrigerated, the components must be returned to the desired operational temperature range, e.g., room temperature, since temperature may affect chemical reactions, e.g., the time required to complete a reaction. Certain parts of the test sensor may return transition to a stabilized temperature at different rates. If the test sensor does not reach a stable temperature prior to taking a reading, the reading may be inaccurate.

A continuing need exists for systems that account for error in readings that may arise due to the changing temperature of the system.

### BRIEF SUMMARY OF THE INVENTION

Devices and methods for reliably and accurately measuring the concentration of target analytes within a fluid sample are described.

A lateral flow fluid analyte device for detecting at least one target analyte in a fluid sample may include at least one test strip including at least one zone in which a first zone has a color having an intensity that is dependent upon a concentration of a target analyte in the fluid sample.

A first temperature sensor may be configured to detect the temperature of the at least one zone. A second temperature sensor may be configured to detect a reference temperature. The functions performed by the first and second temperature sensors may in embodiments be performed by one or more devices, i.e., in embodiments a single device may measure temperature at more than one location. A temperature monitor, e.g., a processor, may analyze the temperature measurements to determine when the temperature at the zones (or at predetermined locations) has stabilized or is substantially stable. Alternatively, once the temperature at the zone is determined, such temperature may be calibrated using an algorithm to standardize the temperature across one or more zones.

Chemical reactions may be temperature dependent. For example, the temperature may affect the time required for a reaction to complete. Therefore, the color intensity of the zones may be affected by temperature and could lead to a false reading if an insufficient amount of time was provided at a given temperature. Therefore, it is desirable to determine whether the temperature in each of the one or more zones has stabilized or is substantially stable and/or whether test is being conducted within an acceptable temperature range. Stabilization of the temperature in each of the one or more zones may be determined by taking the standard deviation of temperatures between the zones of the test strip. A small standard deviation, e.g., zero, would be indicative of a stable, i.e., unchanging, temperature. Thermocouples may be used to determine the temperature in each of the one or more zones. In an embodiment, infrared sensors may be used to determine the temperature in each of the one or more zones. Alternatively, both thermocouples and infrared sensors may be used to determine the temperature in each of the one or more zones.

A fluid analyte system may include a cartridge housing at least one test strip that is configured to measure the amount of particular substances in a fluid, e.g., blood. Placement of the test strip within a cartridge housing facilitates thermal isolation of the test strip from the user's body temperature, e.g., the warmth of the user's fingers, is facilitated.

The cartridge may include a sample well that is configured to receive the fluid, which is drawn through the test strip. When the test strip comes in contact with the fluid deposited in the sample well, the fluid wicks through the test strip. The test strip includes one or more zones that are configured and adapted to provide an indication of the amount of a particular substance in the fluid. The indication may be optical, e.g., color intensity in the zone changes in response to the amount of a particular substance in the fluid. The color may be provided by having the fluid pass through a zone that has microparticles, e.g., colored microparticles, which mix with the fluid before passing through the one or more zones. As reactions occur in zones testing for a target analyte, the colored microparticles are captured or collected within the zone such that the color intensity within the zone corresponds to the concentration of the target analyte within the fluid.

Another embodiment of a lateral flow fluid analyte device is also disclosed that can detect at least one target analyte in a fluid sample. This alternative device may include at least one test strip, a first sensor, a processor and an optical sensor. The test strip may include a first zone in which the first zone has a color that has a reflectance dependent upon a concentration of a target analyte in the fluid sample. The first sensor determines a reference temperature at a reference location on the device. A second sensor at the first zone provides a differential temperature or a difference in temperature between the reference temperature and the temperature at the first zone. The processor determines the temperature at the first zone based upon the difference in temperature between the reference temperature and the temperature at the first zone. An optical sensor at the first zone can be configured to use the temperature at the first zone to determine the reflectance of the color at the first zone.

A second sensor may also extend between the first zone and the reference location. Such second sensor may be a thermocouple. There may also be a second zone on the test strip and a third sensor at the second zone for providing a second differential temperature. The processor can determine the second zone temperature based upon the second differential temperature, i.e., the difference in temperature between the second zone temperature and the reference temperature. The processor can also determine the deviation that exists between the temperature of the first zone and the second zone temperature and make a determination as to when the temperature for the first zone and the second zone temperature are stabilized. A second optical sensor at the second zone can be configured to use the second zone temperature to determine the reflectance of the color at the second zone when the temperature of the first zone and second zone temperature is stabilized.

A method for analyzing one or more substances within a fluid sample is also disclosed. In an embodiment, a lateral flow fluid analyte device is provided. The device may include a sample well and at least one test strip. Each test strip may include a first zone for detecting a substance within the fluid sample, and a second zone for detecting a specific matter within the substance. The first and second zones may each have a color corresponding to the substance and specific matter that comes into contact with the first and second zone respectively. An optical sensor may measure the intensity of color at each zone. The color intensity corresponds to a characteristic being measured, e.g., the concentration of a target analyte. Each zone may include a temperature sensor to measure temperature of that zone. A temperature sensor may measure a reference temperature. The intensity of the color at each zone may be taken when the temperature of the test strip, is within an acceptable range and the standard deviation of the temperatures amongst zones or between corresponding zones, e.g., a first zone on a first strip and a first zone on a second strip, on multiple strips is at an acceptable predetermined valve. When using multiple strips, an average value for the color intensity at corresponding zones may be determined. In an embodiment, a thermocouple may be used to determine temperatures at multiple locations, e.g., zones. In another embodiment, an infrared sensor may be used to determine temperatures at multiple locations, e.g., zones.

These and other embodiments of the present disclosure are described in greater detail hereinbelow.

### BRIEF DESCRIPTION OF THE DRAWINGS

By way of description only, embodiments of the present disclosure will be described herein with reference to the accompanying drawings, in which:

FIG. 1 is a top plan view of a monitoring system shown with a monitor separated from a cartridge having a housing;

FIGS. 2A, 2B, and 2C are a schematic illustration of the use of the monitoring system of FIG. 1;

FIG. 3 is a top plan view of the cartridge of FIG. 1 shown without the top of the cartridge housing and including two test strips;

FIG. 3A is a top plan view of one of the test strips of FIG. 3; and

FIG. 4 is a top plan view of an alternative cartridge shown without the top of the cartridge housing and including two test strips.

### DETAILED DESCRIPTION

Particular embodiments of the present disclosure are described with reference to the accompanying drawings. In the figures and in the description that follow, like reference numerals identify similar or identical elements.

A monitoring system 100 and the use of the monitoring system 100 are described herein with reference to FIGS. 1-4.

The monitoring system 100 includes a monitor 52 including a display 101 and a port 102 for the reception of a cartridge 50 therein. The monitoring system 100 may include a processor (not shown) to collect and calculate measurements. The cartridge 50 may include a housing 53 that includes an aperture 51 to provide access to a sampling well 51. In an embodiment, all the parts of the monitoring system 100 are at the same temperature within a specified range, e.g., 18° C. to 28° C.

As shown in FIGS. 2A-2C, during use blood B is collected from a patient H. A lancet (not shown) or a venous draw (not shown) may be used to draw blood from the patient H. A blood collector 70 may be used to collect the blood B. The blood collector 70 may include a tube 71 that draws the blood therein via capillary action. Once the blood is collected, the blood is diluted with a solution. As shown in FIG. 2B, the blood collector 70 may be coupled to a sampler body 80 that contains a solution such that the blood B mixes with the solution to form a diluted blood sample.

The cartridge 50 can be coupled with the monitor 52. The monitor 52 may provide an indication as to when the monitoring system 100 is ready to receive the diluted blood in the sample well “S”. As shown in FIG. 2C, once the diluted blood sample is placed in the sample well “S”, the monitoring system 100 will analyze the fluid sample to determine the presence of certain analytes or desired information from the sample. The monitoring system 100 may display the results, e.g., the percent A1C in the blood B, within a predetermined amount of time, e.g., within 5 minutes. Thereafter, the cartridge 50 may be discarded, and the monitor 52 re-used at a later time.

As shown in FIG. 1, cartridge 50 may be encased within cartridge housing 53 to facilitate handling of the cartridge 50 and coupling of the cartridge 50 to the monitor 52. The cartridge housing 53 may help to substantially thermally isolate the cartridge 50 from the user during handling of the cartridge 50.

As shown in FIG. 3, the cartridge 50 may include one or more test strips 30A,30B that are configured for use in a lateral flow assay test, such as the test strip disclosed in U.S. Pat. No. 7,439,033, which is assigned to Bayer Healthcare LLC, the disclosure of which is incorporated herein by reference. When the test strips 30A,30B come into contact with a fluid sample, the fluid sample is absorbed by the test strips 30A, 30B and travels in direction x as indicated in FIG. 3A. The test strips 30A, 30B may include a plurality of zones. As shown in FIG. 3A, test strips 30A, 30B include a first zone 1, a second zone 2, and a third zone 3. In one embodiment, each of the zones is a discrete zone. The first zone 1 has colored microparticles, which are configured and adapted to mix with the diluted fluid sample as the fluid sample travels in direction x. It is to be understood that the assay formats discussed herein are intended to be illustrative and are not intended to be limiting. For example, the assay format of zone 1 may be competitive or inhibitive. For example, the colored microparticles be configured to bind with particular substances in the blood or may be configured to resist binding with particular substances in the blood.

In an embodiment, colored microparticles within zone 1 may be configured to interact with particular substances in the blood such that as the blood travels through the test strip, the concentration of microparticles within zone 1 may change. For example, as blood travels through the test strip, hemoglobin A1c within the blood may bind to the colored microparticles and the concentration of colored microparticles within zone 1 may therefore be reduced such that the color of zone 1 will correspondingly change. For example, if the colored microparticles are blue zone 1 may become less blue as hemoglobin A1c in the blood binds to the microparticles and is drawn out from zone 1 as the blood continues to wick through the test strip.

The second zone 2 and the third zone 3 may be configured and adapted to react with specific substances in the blood. For example, second zone 2 may include a substance that interacts with hemoglobin present in the blood. In an embodiment, the substance in zone 2 may include ferricyanide and cyanide. The ferricyanide oxidizes the iron in the hemoglobin, thereby changing hemoglobin to methemoglobin. The methemoglobin unites with the cyanide to form cyanmethemoglobin, which produces a color that is measurable. For example, a light source, e.g., light emitting diode, may emit a light optimized for a particular color and the reflectance may be measured. Since the measured color of the cyanmethemoglobin corresponds to the concentration of hemoglobin in the blood, the concentration of hemoglobin in the blood may therefore be determined. By taking the ratio between the hemoglobin A1c measured in zone 1 to total hemoglobin measured in zone 2, the percent of A1c in the blood may be determined.

The amount or concentration of colored microparticles captured in each of zones 2, 3, may correspond to the amount of the particular substance in the blood. For example, as the diluted blood travels in direction x through the test strip 30, the diluted blood mixes with the colored microparticles in zone 1. In one example, the colored microparticles are captured in zone 2 that correspond with the amount of HbA1C in the sample and colored microparticles may be captured in zone 3 to correspond with the amount of total Hb. The intensity of the color in any of the particular zones corresponds with the amount of colored microparticles that are captured in the zones. By measuring the intensity of the color in zone 2 and zone 3, the concentration of particular substances is determined. Thus, the greater the intensity of color in zone 2 or zone 3, the greater the captured HbA1C in zone 2 and the greater the total Hb captured in zone 3. Thus, the estimated % A1C value may be determined as a function of reflectance from zone 2 and zone 3. By using a plurality of test strips 30A, 30B average measurements may be taken to facilitate a more accurate measurement. In one embodiment, two test strips may be used to average measurements, but more than two may also be used. It is to be appreciated that any known methods or test strips may be utilized to obtain a reaction between an analyte or substance in a fluid sample. Moreover, identifying an analyte or substance based upon the measurement of reflectance based upon captured colored microparticles is just one example.

Accuracy of the measurements is affected by several factors including temperature and time. It is desirable that the colored microparticles substantially or fully conjugate at zones 2 and 3 to accurately reflect the amount of the particular substance being measured. An optical sensor (not shown) may be provided at each of zones 2 and 3 to measure the reflectance. The time required for a reaction to complete is a function of temperature. Thus, to facilitate completion of the test within an acceptable amount of time, e.g., ranging between 3-7 minutes, it is desirable to perform the test within a certain temperature range.

In addition, it has been determined that the color reflectance in zone 2 and zone 3 may be temperature dependent, e.g, the time for the reaction in the zone to complete is a function of temperature. Thus, it is desirable that the temperature in zones 2, 3 be stable such that the reflectance or intensity of the color is likewise stable. Various methods and devices may be utilized to determine whether the temperature in zones 2, 3 is stable. The differential temperatures between each of the zones 1, 2, 3 on one or more strips 30 may be taken such that the standard deviation between the temperatures may be determined. When the standard deviation between the zones is at or near zero, the temperature of each of the zones 1, 2, 3 can be assumed to be constant. A processor can be used to determine the standard deviation.

In an embodiment, the standard deviation may be taken across all of the zones 1, 2, 3 or just at those zones at which color intensity is being measured, i.e., zones 2 and 3. In another embodiment, the standard deviation between corresponding zones 1, 2, 3 on multiple strips 30 may be determined, e.g., zone 1 on a first strip as compared to zone 1 on a second strip. Alternatively, or additionally, the temperature of each of the zones 1, 2, 3 may be continually monitored over a given time period or at intervals over a given time period. When the temperature reading at each of the zones 1, 2, 3 ceases to change, it can be assumed that the temperature reading is stable. It is contemplated that color readings over a length of time may be compared to determine when the color within a given zone 2, 3 at which color intensity is measured has stabilized.

The temperature of each of the zones 1, 2, 3 may be determined using thermocouples or their equivalent. Thermocouples 36, 38, 40, 42, 44, 46 and 48 are differential temperature measurement devices constructed from two wires that are made from dissimilar metals. One wire is pre-designated as the positive side, and the other as the negative.

In this embodiment, thermocouples 36, 38, 40, 42, 44, 46 and 48 can be utilized to help determine the actual temperature in each zone 1, 2, 3 and thermocouple 48 can be used to help determine the temperature of the sample well S. For example, as shown in FIG. 3C, a schematic representation of a cartridge base and test strips 30A,30B provided thereon, a series of thermocouples 36, 38, 40, 42, 44, 46 and 48 are provided on the inside of the cartridge base 50. Thermocouples 36, 38, 40, 42, 44, 46 and 48 may be printed onto the cartridge base, but any known methods of providing thermocouples on cartridge base 49 may be used. In this embodiment, when the base 49 of cartridge 50 is joined together with the top (not shown) of cartridge 50, thermocouples 36, 38, 40, 42, 44, 46 and 48 on base 49 of cartridge 50 will contact test strips 30A,30B.

As shown, thermocouple 36 has a first open end 36a, a second open end 36b, and third closed end 36c. The two wires comprising thermocouple 36 are joined together to form a hot (measuring) junction 37H at third closed end 36c. Hot junction 37H is a junction of dissimilar metals, which can produce an electric potential related to temperature and provide the temperature at zone 3. While the third closed end 36c of thermocouple 36 is positioned at hot junction 37H, first open end 36a and second open end 36b of thermocouple 36 are both positioned at the cold junction “C.” A bend in thermocouple 36 along its length allows for the direction of thermocouple 36 to change, so as to allow for a direct connection between zone 3 and cold junction C. As shown, cold junction C is located between the lengths of each of the test strips. In this embodiment, cold junction C is centrally located between the two test strips. Examples of thermocouples and commercially available thermoucouples that can be implemented in connection with the present embodiments are more fully discussed in The Omega Temperature Measurement Handbook® and Encyclopedia, Vol. MMXIV™ 7th Edition and in Unsheathed Fine Gage Thermocouples, at http://www.omega.com/Temperature/pdf/IRCO_CHAL_P13R_P10R.pdf (last visited Mar. 11, 2013), the disclosures of which is incorporated herein by reference.

The remaining thermocouples 38, 40, 42, 44, 46 and are each identical to thermocouple 36, except that the lengths of the respective thermocouples may differ based upon the location of the respective hot junctions. Thermocouples 38, 40, 42, 44, 46 and 48 each have respective first open ends 38a, 40a, 42a, 44a, 46a and 48a; respective second open ends 38b, 40b, 42b, 44b, 46b and 48b; and respective third closed ends 38c, 40c, 42c, 44c, 46c and 48c. The two metals comprising each of the thermocouples 36, 38, 40, 42, 44, 46 and 48 are joined at respective third closed ends 36c, 38c, 40c, 42c, 44c, 46c and 48c to form a hot (measuring) junction 37H, 39H, 41H, 43H, 45H, 47H and 49H. The respective first open ends 38a, 40a, 42a, 44a, 46a and 48a and respective second open ends 38b, 40b, 42b, 44b, 46b and 48b form a reference portion 37R, 39R, 41R, 43R, 45R, 47R and 49R. In other words, each reference portion 37R, 39R, 41R, 43R, 45R, 47R and 49R is at the end of thermocouples 36, 38, 40, 42, 44, 46 and 48 that is opposite to respective hot junctions 37H, 39H, 41H, 43H, 45H, 47H and 49H.

In one embodiment, each reference portion 37R, 39R, 41R, 43R, 45R, 47R and 49R of each thermocouple 36, 38, 40, 42, 44, 46 and 48 is joined together at a common place. As shown, each reference portion 37R, 39R, 41R, 43R, 45R, 47R and 49R (i.e., the first ends 36a, 38a, 40a, 42a, 44a, 46a and 48a of each respective thermocouple, as well as the corresponding second ends 36b, 38b, 40b, 42b, 44b, 46b and 48b or each respective thermocouple) is positioned at the cold junction C of the cartridge, such that the cold junction C provides for a common junction or point where reference portion 37R, 39R, 41R, 43R, 45R, 47R and 49R join together. As shown, thermocouples 38, 40, 42, 44, 46 join each of zones 1, 2, 3 and a cold junction “C”. Thermocouples 36,46 join cold junction C with zone 3; thermocouples 38,44 join cold junction “C” with zone 2; and thermocouples 40,42 join cold junction C with zone 1. Thermocouple 48 joins cold junction C with sample well S.

The cold junction C provides a reference temperature for each of the thermocouples, such that the reference portions 37R, 39R, 41R, 43R, 45R, 47R and 49R are at the same temperatures. The voltage between the respective hot junctions 37H, 39H, 41H, 43H, 45H, 47H and 49H and the temperature of the cold junction C can provide for a differential temperature between the cold junction C and each of the respective hot junctions 37H, 39H, 41H, 43H, 45H, 47H and 49H and.

It is to be appreciated that the thermocouples may take on alternative configurations to determine temperature differentials. By way of one example, in one alternative embodiment, each of the first ends 36a, 38a, 40a, 42a, 44a, 46a and 48a second ends 36b, 38b, 40b, 42b, 44b, 46b and 48b of the respective thermocouples 36, 38, 40, 42, 44, 46 and 48 may be connected or joined together at their open ends to form a second junction. The second junction would be a reference junction at the end of the thermocouple directly opposite the hot junction.

To determine the hot junction temperature, i.e., the temperature at each zone 1, 2, 3, the temperature of cold junction C is first determined through any suitable means for temperature measurement, e.g., infrared (IR) sensor. A voltage is then measured between the hot junction 37C, 39H, 41H, 43H, 45H, 47H and 49H of each thermocouple 36, 38, 40, 42, 44, 46 and 48 and the cold junction C. For example, the voltage been hot junction 37 and the cold junction C is determined; the voltage been hot junction 46 and cold junction C is determined; and the voltage between hot junction 49 and cold junction C is determined. Since the voltage is a function of the difference between the temperatures of the cold junction C and the hot junction, the temperature of the hot junction can be readily determined when the temperature of the cold junction C is known. In an alternative embodiment, the temperature may be read at one or more zones 1, 2, 3 and cold junction C by using an IR sensor.

When the temperature of the test strip at each zone 1, 2, 3 is not within an acceptable range and the standard deviation of temperature between zones 1, 2, 3 (or among the desired zones) is not at an acceptable value, the time duration of the test may be altered, e.g., lengthened, or an error message may be displayed on the monitor 101 if the test cannot be performed within an acceptable amount of time. When the conditions, i.e., the temperature of the test strip and standard deviation of temperature between zones 1, 2, 3 of the one or more test strips 30, are met, the color intensity at the zones 1, 2, 3, at which a reading or measurement is desired is taken. By taking the reading when such conditions are met, the possibility of an erroneous reading is minimized.

The readings at each of the zones 1, 2, 3 may be used to calculate or infer additional measurements. In an embodiment, zone 1 may be HbA1C specific and zone 2 may measure the total Hb, and thus estimated % A1C is a function of the reflectance from zone 1 and zone 2. It is contemplated that further characteristics may be calculated or inferred from readings that are taken by the device. Turning now to FIG. 4, a top plan view of an alternative embodiment of a test cartridge 150 utilizing thermocouples is shown. In this embodiment, a first test strip 130A and a second test strip 130B are positioned on the cartridge base 149, each test strip 130A, 130B having a first, second and third zone 101,102, and 103. Test strips 130A, 130B meet at a point 111. As shown, cold junction C is located between the ends 110 of test strips 30A,30B, and the sample well S is spaced a certain distance away from cold junction C. Cold junction C and sample well S are positioned closer together, as compared to the previous embodiment. The location of the cold junction C between the ends 110 of test strips 30A, 30B allows for thermocouples to extend along the length of the test cartridge, as opposed to the previous embodiment, in which the thermocouples extend away from the test strip. The location of the cold junction C also shortens the run of the respective thermocouples and allows the thermocouples to run in only one direction, so as to eliminate the need for the thermocouples to “bend” or change direction.

Like the previous embodiment, each of the thermocouples has first ends 136a, 138a, 140a, 142a, 144a, 146a and 148a and second ends 136b, 138b, 140b, 142b, 144b, 146b and 148b that are both positioned at cold junction C. The thermocouples also have respective hot junctions 137H, 139H, 141H, 143H, 145H, 147H and 149H at their respective third ends 138c, 140c, 142c, 144c, 146c and 148c. Thermocouples 136, 138, 140, 142, 144, 146 join each of the zones 1, 2, 3 and cold junction C. Thermocouple 148 joins sample well S to the cold junction C.

When the cartridge is inserted into a meter, the testing can be carried out as discussed in the previous embodiment. The temperature of the cold junction C is first determined through any suitable means for temperature measurement, e.g., infrared (IR). To determine the hot junction temperature, i.e., the temperature at each zone 1, 2, 3, the temperature of cold junction C is first determined. A voltage is measured between the hot junction and the cold junction C. Since the voltage is a function of the difference between the temperatures of the cold junction C and the hot junction, the temperature of the hot junction can be readily determined when the temperature of the cold junction C is known. In an embodiment, temperature may be read at one or more zones 1, 2, and 3 by using an IR sensor.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims. For example, it is to be understood that the devices described herein may employ any assay format as is known in the art including, for example, a competitive and/or inhibitive assay format.

1. A lateral flow fluid analyte device for detecting at least one target analyte in a fluid sample, the device comprising:
at least one test strip including at least one zone in which a first zone has a color having an intensity that is dependent upon a concentration of a target analyte in the fluid sample;
a first temperature sensor that is configured to detect a temperature of the at least one zone; and
a temperature monitor configured to determine when the temperature of the at least one zone has substantially stabilized.

2. The device of claim 1, further comprising a second temperature sensor that is configured to detect a reference temperature.

3. The device of claim 1, wherein the at least one test strip includes a plurality of zones.

4. The device of claim 3, wherein the temperature monitor determines a standard deviation among the temperatures detected at the zones.

5. The device of claim 4, wherein a standard deviation equal to zero is indicative that the temperatures at the zones have stabilized.

6. The device of claim 1, wherein the device comprises at least two test strips.

7. The device of claim 1 further comprising an optical sensor to detect the color of the at least one zone.

8. The device of claim 1 further comprising a sample well configured to receive the fluid sample, and wherein the test strip is configured to absorb the fluid sample such that the fluid sample travels to the at least one zone.

9. The device of claim 1, wherein the test strip includes a second zone including colored microparticles that are configured to uniformly mix with the fluid sample.

10. The device of claim 9, wherein the colored microparticles are collected at the first zone, the amount of colored microparticles collected at the first zone corresponding to the concentration of the target analyte in the fluid sample.

11. The device of claim 1, wherein the first temperature sensor includes a thermocouple.

12. The device of claim 1, wherein the first temperature sensor includes an infrared sensor.

13. The device of claim 1 further comprising a cartridge housing, wherein the at least one test strip is housed within the cartridge housing.

14. A lateral flow fluid analyte device for detecting at least one target analyte in a fluid sample, the device comprising:
at least one test strip including a first zone in which the first zone has a color having a reflectance that is dependent upon a concentration of a target analyte in the fluid sample;
a first sensor for determining a reference temperature at a reference location on the device;
a second sensor at the first zone for providing a differential temperature between the reference temperature and a temperature at the first zone;
a processor for determining the temperature at the first zone based upon the differential temperature; and
an optical sensor at the first zone, the optical sensor configured to use the temperature at the first zone to determine the reflectance of the color at the first zone.

15. The device of claim 14, wherein the second sensor extends between the first zone and the reference location.

16. The device of claim 14, wherein the second sensor is a thermocouple.

17. The device of claim 14, further comprising a second zone on the test strip and a third sensor at the second zone for providing a second differential temperature,
the processor determining the second zone temperature based upon the differential temperature between the second zone temperature and the reference temperature;
the processor comparing a deviation between the temperature of the first zone and the second zone temperature to determine when the temperature of the first zone and the second zone temperature are stabilized;
a second optical sensor at the second zone, the optical sensor configured to use the second zone temperature to determine the reflectance of the color at the second zone when the temperature of the first zone and second zone temperature are stabilized.

18. A method for analyzing one or more substances within a fluid sample comprising:
providing lateral flow fluid analyte device comprising:
at least one test strip, each test strip configured to receive a fluid sample, each test strip including a first zone for detecting a substance within the fluid sample, and a second zone for detecting a specific matter within the substance, wherein the first and second zones each has a color corresponding to the substance and specific matter that comes into contact with the first and second zone respectively;
an optical sensor at each zone to measure an intensity of the color of each zone;
a first temperature sensor at each of the zones configured to measure the temperature of the zone; and
a second temperature sensor configured to measure a reference temperature;
placing a fluid sample within the sample well;
allowing the fluid sample to wick through the test strips, thereby causing the first and second zones to change color;
measuring the temperature of each zone;
determining a standard deviation either among the temperatures measured at each of the zones or between corresponding zones on multiple test strips;
measuring the reference temperature; and
measuring the intensity of the color of each zone when the reference temperature is within a predetermined range and when the standard deviation is at a predetermined value.

19. The method of claim 18, wherein at least one of the first and second sensors includes a thermocouple.

20. The method of claim 18, wherein at least one of the first and second sensors includes an infrared sensor.

21. The method of claim 18, wherein the device further comprises a cartridge housing, and wherein the test strips are housed within the cartridge housing.

22. The method of claim 18 may further include determining an average value of the intensity of the color at corresponding zones on multiple strips.
